(19) United States
(12) Patent Application Publication
(10) Pub. No.: US 2025/0210138 A1
(43) Pub. Date: Jun. 26, 2025

(54) METHOD, DEVICE AND MEDIUM FOR CONFORMATION GENERATION OPTIMIZATION

(71) Applicants: Bytedance Technology Ltd., Grand Cayman (KY); Beijing Youzhuju Network Technology Co., Ltd., Pinggu District (CN)

(72) Inventors: Yuning Shen, Los Angeles, CA (US); Lihao Wang, Beijing (CN); Huizhuo Yuan, Los Angeles, CA (US); Yan Wang, Beijing (CN); Bangji Yang, Beijing (CN); Quanquan Gu, Los Angeles, CA (US)

(21) Appl. No.: 19/074,013

(22) Filed: Mar. 7, 2025

(51) Int. Cl. G16B 30/00

(52) U.S. Cl. CPC G16B 30/00

(57) ABSTRACT

Embodiments of the present disclosure provide a solution for conformation generation optimization. A method includes: determining a sequence feature representation and a conformation feature representation of a protein, the sequence feature representation characterizing an amino acid sequence of the protein and the conformation feature representation characterizing a conformation of the protein; fusing the sequence feature representation and the conformation feature representation based on a plurality of reference conformations in a conformation sequence of the protein, to obtain a fused feature representation; and generating a target conformation at a target temporal position in the conformation sequence at least based on the fused feature representation.

### FIELD

The present disclosure generally relates to computer technologies, and more specifically, to a method, apparatus, device and computer readable storage medium for conformation generation optimization.

### BACKGROUND

Proteins are flexible molecules capable of transitioning between multiple structural states, known as conformations. This ability allows them to perform a wide range of functions critical to life, including ligand binding, transmembrane signaling, and catalyzing reactions. Characterizing protein conformational dynamics is the key to understanding protein behavior, predicting its function, and designing novel proteins.

### SUMMARY

In a first aspect of the present disclosure, there is provided a method of conformation generation optimization. The method comprises: determining a sequence feature representation and a conformation feature representation of a protein, the sequence feature representation characterizing an amino acid sequence of the protein and the conformation feature representation characterizing a conformation of the protein; fusing the sequence feature representation and the conformation feature representation based on a plurality of reference conformations in a conformation sequence of the protein, to obtain a fused feature representation; and generating a target conformation at a target temporal position in the conformation sequence at least based on the fused feature representation.

In a second aspect of the present disclosure, there is provided an apparatus for conformation generation optimization. The apparatus comprises: a determining module configured to determine a sequence feature representation and a conformation feature representation of a protein, the sequence feature representation characterizing an amino acid sequence of the protein and the conformation feature representation characterizing a conformation of the protein; an fusing module configured to fuse the sequence feature representation and the conformation feature representation based on a plurality of reference conformations in a conformation sequence of the protein, to obtain a fused feature representation; and a generating module configured to generate a target conformation at a target temporal position in the conformation sequence at least based on the fused feature representation.

In a third aspect of the present disclosure, there is provided an electronic device. The electronic device comprises: at least one processor; and at least one memory coupled to the at least one processor and storing instructions executable by the at least one processor, the instructions, upon execution by the at least one processor, causing the electronic device to perform: determining a sequence feature representation and a conformation feature representation of a protein, the sequence feature representation characterizing an amino acid sequence of the protein and the conformation feature representation characterizing a conformation of the protein; fusing the sequence feature representation and the conformation feature representation based on a plurality of reference conformations in a conformation sequence of the protein, to obtain a fused feature representation; and generating a target conformation at a target temporal position in the conformation sequence at least based on the fused feature representation.

In a fourth aspect of the present disclosure, a computer-readable storage medium is provided. The computer-readable storage medium stores computer executable instructions which, when executed by an electronic device, causes the electronic device perform operations comprising: determining a sequence feature representation and a conformation feature representation of a protein, the sequence feature representation characterizing an amino acid sequence of the protein and the conformation feature representation characterizing a conformation of the protein; fusing the sequence feature representation and the conformation feature representation based on a plurality of reference conformations in a conformation sequence of the protein, to obtain a fused feature representation; and generating a target conformation at a target temporal position in the conformation sequence at least based on the fused feature representation.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE SEVERAL VIEWS OF THE DRAWINGS

The above and other features, advantages and aspects of the embodiments of the present disclosure will become more apparent in combination with the accompanying drawings and with reference to the following detailed description. In the drawings, the same or similar reference symbols refer to the same or similar elements, where:

FIG. 1 illustrates a schematic diagram of an example environment in which embodiments of the present disclosure may be implemented;

FIG. 2 illustrates a schematic diagram of an example architecture of the conformation generation model 110 according to some embodiments of the present disclosure;

FIG. 3A illustrates a schematic diagram of an architecture of an auto regressive model in accordance with some embodiments of the present disclosure;

FIG. 3B illustrates a schematic diagram of another architecture of an auto regressive model in accordance with some embodiments of the present disclosure;

FIG. 4 illustrates a flowchart of a method of conformation generation optimization in accordance with some example implementations of the present disclosure;

FIG. 5 shows a block diagram of an apparatus for conformation generation optimization in accordance with some embodiments of the present disclosure; and

FIG. 6 illustrates a block diagram of an electronic device in which one or more embodiments of the present disclosure can be implemented.

### DETAILED DESCRIPTION

The embodiments of the present disclosure will be described in more detail below with reference to the accompanying drawings. Although some embodiments of the present disclosure are shown in the drawings, it would be appreciated that the present disclosure may be implemented in various forms and should not be interpreted as limited to the embodiments described herein. On the contrary, these embodiments are provided for a more thorough and complete understanding of the present disclosure. It would be appreciated that the drawings and embodiments of the present disclosure are only for the purpose of illustration and are not intended to limit the scope of protection of the present disclosure.

In the description of the embodiments of the present disclosure, the term “including” and similar terms would be appreciated as open inclusion, that is, “including but not limited to”. The term “based on” would be appreciated as “at least partially based on”. The term “one embodiment” or “the embodiment” would be appreciated as “at least one embodiment”. The term “some embodiments” would be appreciated as “at least some embodiments”. Other explicit and implicit definitions may also be included below. As used herein, the term “model” can represent the matching degree between various data. For example, the above matching degree can be obtained based on various technical solutions currently available and/or to be developed in the future.

It will be appreciated that the data involved in this technical proposal (including but not limited to the data itself, data acquisition or use) shall comply with the requirements of corresponding laws, regulations and relevant provisions.

It will be appreciated that before using the technical solution disclosed in each embodiment of the present disclosure, users should be informed of the type, the scope of use, the use scenario, etc. of the personal information involved in the present disclosure in an appropriate manner in accordance with relevant laws and regulations, and the user's authorization should be obtained.

For example, in response to receiving an active request from a user, a prompt message is sent to the user to explicitly prompt the user that the operation requested operation by the user will need to obtain and use the user's personal information. Thus, users may select whether to provide personal information to the software or the hardware such as an electronic device, an application, a server or a storage medium that perform the operation of the technical solution of the present disclosure according to the prompt information.

As an optional but non-restrictive implementation, in response to receiving the user's active request, the method of sending prompt information to the user may be, for example, a pop-up window in which prompt information may be presented in text. In addition, pop-up windows may also contain selection controls for users to choose “agree” or “disagree” to provide personal information to electronic devices.

It will be appreciated that the above notification and acquisition of user authorization process are only schematic and do not limit the implementations of the present disclosure. Other methods that meet relevant laws and regulations may also be applied to the implementation of the present disclosure.

As used herein, the term “model” is referred to as an association between an input and an output learned from training data, and thus a corresponding output may be generated for a given input after the training. The generation of the model may be based on a machine learning technique. In general, a machine learning model may be built, which receives input information and makes predictions based on the input information. For example, a classification model may predict a class of input information among a predetermined set of classes. As used herein, “model” may also be referred to as “machine learning model”, “learning model”, “machine learning network”, or “learning network,” which are used interchangeably herein.

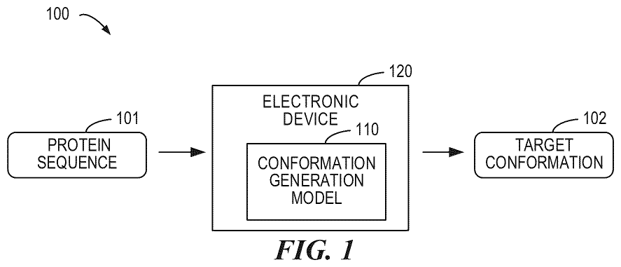

FIG. 1 illustrates a block diagram of an example environment 100 in which various embodiments of the present disclosure may be implemented. In the environment 100 of FIG. 1, a conformation generation model 110 is deployed in an electronic device 120. The electronic device 120 receives a protein sequence (e.g., chemical composition sequence) 101 of a target protein. The electronic device 120 uses the conformation generation model 110 to generate a target conformation 102 of the target protein based on the protein sequence 101.

In some embodiments, the electronic device 120 may also receive one or more reference conformations of the target protein as a part of trajectory to predict the target conformation 102.

In the environment 100 of FIG. 1, the electronic device 120 may include any computing system with computing capability, such as various computing devices/systems, terminal devices, servers, etc. Terminal devices may include any type of mobile terminals, fixed terminals, or portable terminals, including mobile phones, desktop computers, laptops, netbooks, tablets, media computers, multimedia tablets, or any combination of the aforementioned, including accessories and peripherals of these devices or any combination thereof. Servers include but are not limited to mainframe, edge computing nodes, computing devices in cloud environment, etc.

As mentioned above, understanding conformational dynamics is crucial for studying the structural and functional properties of proteins. Simulation-based methods, such as molecular dynamics (MD), are conventional ways for studying protein conformational changes. These simulations use physical models to describe the energy of a protein's structure and the forces acting on its atoms. Protein motion follows classical mechanics, and by running simulations over time, researchers can explore different conformations, track how proteins transition between states, and gain insights into their behavior. However, thoroughly exploring a protein's conformation landscape is both costly and challenging. A major limitation stems from the large gap between the small simulation time steps (10-15 seconds) and the biologically relevant timescales of motion (10⁻⁶˜1 seconds).

Furthermore, the simulation process often becomes trapped in local energy minima, making it difficult to overcome the energy barriers and reach another conformation state. These challenges limit the scalability of MD simulations for biological research and drug discovery. Recent research has explored learning protein conformational dynamics from trajectory data generated from large-scale MD datasets. An MD trajectory is a sequence of time-dependent conformational frames, x^{1:L}, where each frame x^{l} is sampled based on previous frames x^{<l}. This process naturally aligns with an autoregressive generation process: q(x^{1:L})=Πₗ₌₁^{L}q (x^{l}|x^{<l}), where q(x^{1:L}) represents the joint probability of the trajectory and x^{<l} denotes the previous frames. Conventional machine learning models either learn a forward transport operator to predict a future frame from current frame q(x^{l+Δl}|x^{l}) which is a special case of single-history autoregressive model or jointly generate trajectories q(x^{1:L}| [symbol] ), with condition [symbol] being key frames or some predetermined history windows.

In view of the above, there are several technical problems to be solved. Current deep learning models for conformational dynamics can only generate conformations in one of the following ways: autoregressive generation of conformations in a sequential order with predefined history windows, flexible generation (e.g., a trajectory between two conformation states) using non-autoregressive models or generating time-independent conformation samples using a non-dynamic model. In addition, autoregressive models are based on language modeling frameworks that commonly model discrete distributions.

Embodiments of the present disclosure propose an improved solution for conformation generation optimization. In this solution, a sequence feature representation and a conformation feature representation of a protein are determined. The sequence feature representation characterizes an amino acid sequence of the protein and the conformation feature representation characterizes a conformation of the protein. The sequence feature representation and the conformation feature representation are fused based on a plurality of reference conformations in a conformation sequence of the protein, to obtain a fused feature representation. Then, a target conformation is generated at a target temporal position in the conformation sequence at least based on the fused feature representation.

With these embodiments of the present disclosure, the target conformation may be generated based on reference conformations preceding or succeeding the target conformation. In this way, different kinds of conformation generations may be enabled, such as sequential-order conformation generation, flexible-order conformation generation, time-independent conformation generation and the like.

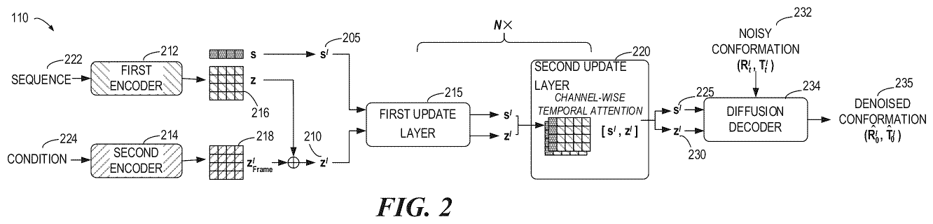

Reference is now made to FIG. 2, which illustrates a schematic diagram of an example architecture of the conformation generation model 110 according to some embodiments of the present disclosure. As illustrated, a sequence feature representation 205 (denoted as s^{l}) and a conformation feature representation 210 (denoted as z^{l}) of a protein (not shown). The sequence feature representation 205 characterizes an amino acid sequence of the protein and the conformation feature representation 210 characterizes a conformation of the protein.

A protein whose chemical and structural information may be encoded as a sequence-dependent prior condition. A trajectory including L protein frames may be modeled through its joint probability distribution as follows:

$$q ( {x}^{1 : L} \vert ) = {\prod }_{l = 1}^{L} q ( {x}^{l} \vert {x}^{< l} , ) \qquad ( 1 )$$

When L=1, Eq. (1) reduces to a single frame distribution q(x| [symbol] ), enabling the direct generation of time-independent conformation samples. Eq. (1) transforms trajectory generation into a next-conformation prediction problem as follows:

$$q ( {x}^{l} \vert {x}^{< l} , ) = {\int }_{h} q ( {x}^{l} \vert {h}^{l} ) \cdot q ( {h}^{l} \vert {h}^{< l} ) \cdot q ( {h}^{< l} \vert {x}^{< l} , ) \qquad ( 2 )$$

where h represents an intermediate latent state. An encoder network may be employed to model q(h^{l}| [symbol] ) and an autoregressive language model may be employed to model q(h^{l}|h^{<l}), both of which are deterministic. Therefore, Eq. (2) may be rewritten as follows:

$$q ( {x}^{l} \vert {x}^{< l} , ) = q ( {x}^{l} \vert {AR}_{ϕ} ( {Encoder}_{ψ} ( {x}^{< l} , ) ) ) \qquad ( 3 )$$

The goal becomes finding a model to jointly learn the family of distributions as follows:

$${p}_{θ} ( {x}^{l} \vert {AR}_{ϕ} ( {Encoder}_{ψ} ( {x}^{< l} , ) ) ) \approx q ( {x}^{l} \vert {x}^{< l} , ) \qquad ( 4 )$$

After determining the sequence feature representation 205 and the conformation feature representation 210, the sequence feature representation 205 and the conformation feature representation 210 may be fused based on a plurality of reference conformations in a conformation sequence (also referred to as trajectory) of the protein, to obtain a fused feature representation. The plurality of reference conformations (also referred to as conditioning set, denoted as [symbol] ={x^{k}}ₖ₌₁^{K}) represents K frames (i.e., conformations) in the conformation sequence, which serve as initial conditions for generating the remaining L frames. The order of the plurality of reference conformations within the conformation sequence may be arbitrary, thereby allowing flexibility in different generation scenarios. In this way, various generation tasks may be achieved, such as forward simulation (by providing the first frame) and trajectory interpolation (by providing both the first and last frames). Conditioned on the plurality of reference conformations, the joint probability distribution for generating the remaining frames is given by q(x^{1:L}| [symbol] [symbol] ). Eq. (1) to (4) may be modified to incorporate the conditioning set [symbol] , enabling the model to adapt to various generation tasks.

Then, a target conformation is generated at a target temporal position in the conformation sequence at least based on the fused feature representation. In some examples, the fused feature representation may include a first fused feature representation 225 corresponding to the sequence feature representation 205 and a second fused feature representation 230 corresponding to the conformation feature representation 210. In some examples, the target temporal position may be a temporal position other than reference temporal positions of the plurality of reference conformations. For example, if the reference temporal positions of the plurality of reference conformations are 1, 3, 5, 7, 9, then the target temporal position may be within the reference temporal positions (e.g., 4, 6, etc.) or after the reference temporal positions (e.g., 10).

In some embodiments, a first conformation feature 216 (denoted as z) may be generated, using a first encoder 212 (also referred to as folding module), based on an amino acid protein sequence 222 corresponding to the sequence feature representation 205. A second conformation feature 218 (denoted as Z_{frame}^{l}) may be generated, using a second encoder 214 (also referred to as frame encoder), from a condition 224 for generating the target conformation.

In some embodiments, the condition 224 may include one of: at least one reference conformation in the plurality of reference conformations that is at a position before the target temporal position or a masked conformation. For example, if the reference temporal positions of the plurality of reference conformations are 1, 3, 5, 7, 9 and the target temporal position is 4, the condition 224 may include reference conformations at temporal positions 1 and 3. The masked conformation does not include any conformation information. For example, by setting the distances between residue pairs as zero values to remove the conformation information, the reference conformation at temporal position 7 may be converted into a masked conformation.

In some embodiments, the first encoder 212 and the second encoder 214 may be constructed based on a folding model. The first encoder 212 may extract the protein-level prior knowledge from the amino acid protein sequence 222. In some examples, the first encoder 212 may be parameterized by using a pretrained OpenFold model to obtain the single (i.e., the sequence feature representation 205) and pair representation (i.e., the sequence feature representation 205 and the first conformation feature 216) for the protein [symbol] =(s∈ [symbol] ^{N×d}^{ₛ}, z∈ [symbol] ^{N×N×d}^{_{z}}), where N denotes the length of the protein, dₛ denote the embedding size for single representation and d_{z} denote the embedding size for the pair representation.

In some examples, the second encoder 214 may extract structural information of each conformation in the condition 224. The second encoder 214 may be employed to first extract the distance between pairs of residues using pseudo-beta-carbon coordinates and then pass through layers of triangular reasoning among these residue pairs. The second encoder 214 encodes the conformation as an SE(3) invariant embedding share the same shape as the pair representation {z_{Frame}^{l}}ₗ₌₁^{L}, z_{Frame}^{l} ∈ [symbol] ^{N×N×d}^{_{z}}. Then, the conformation feature representation 210 may be determined by combining the first conformation feature 216 and the second conformation feature 218.

In some embodiments, the sequence feature representation 205 and the conformation feature representation 210 may be fused by a plurality of first update layers 215 (also referred to as structural update layer) and a plurality of second update layers 220 (also referred to as temporal update layer). The sequence feature representation and the conformation feature representation may be fused, by the plurality of first update layers 215, based on structural relations within each conformation in the plurality of reference conformations and the target conformation. The sequence feature representation and the conformation feature representation may be fused, by the plurality of second update layers 220, the sequence feature representation and the conformation feature representation based on temporal relations across the plurality of reference conformations. In some examples, the plurality of first update layers 215 and the plurality of second update layers 220 may reason the structural and temporal relations across the plurality of reference conformations and reason the structural and temporal relations across the conformations to be generated.

In some embodiments, the plurality of first update layers 205 and the plurality of second update layers 210 may be arranged in an interleaving pattern. For example, one first update layer is arranged firstly, one second update layer is deployed secondly, another first update layer is arranged thirdly and so on. In this way, the sequence feature representation 205 and the conformation feature representation 210 may be fused efficiently.

In some embodiments, the structural relations may indicate spatial positional relationships between different elements within a reference conformation, such as atom A is to the left of atom B. Additionally, the structural relations may also indicate semantic relationships between different elements within a reference conformation, such as atom A is a part of molecule B. The temporal relations may indicate trajectories of elements over time, such as atom A moves from left to right.

In some embodiments, the plurality of first update layers 215 may leverage pairformer layers with triangular operations to directly reason across residue pairs and fuse the sequence feature representation 205 and the conformation feature representation 210.

In some embodiments, by a second update layer of the plurality of second update layers 220, an attention mechanism may be applied to the plurality of reference conformations along temporal dimension of the conformation sequence, to fuse the sequence feature representation 205 and the conformation feature representation 210. At least one reference conformation, of the plurality of reference conformation, that is after the target temporal position may be masked. For example, if the reference temporal positions of the plurality of reference conformations are 1, 3, 5, 7, 9 and the target temporal position is 4, then reference conformations at temporal positions 5, 7 and 9 are masked. To integrate information from other conformations, each second update layer may apply channel-wise self-attention along the temporal dimension of the conformation sequence to fuse the sequence feature representation 205 and the conformation feature representation 210. The relative temporal positions of the plurality of reference conformations may be encoded through rotary position embedding through attention.

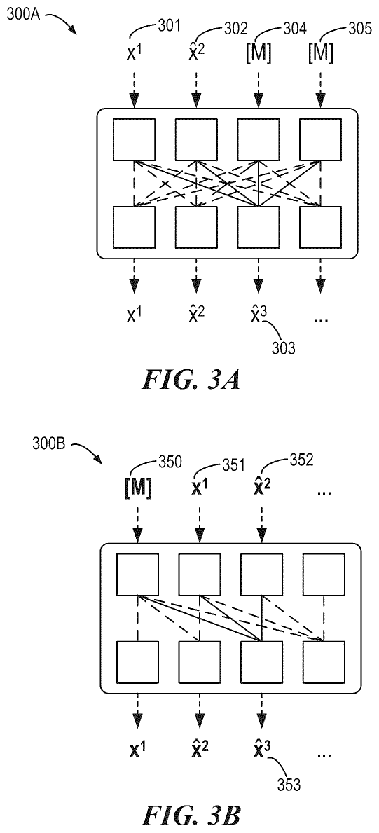

In some embodiments, the second update layer may be constructed based on a masked-style auto regressive model which will be introduced with reference to FIG. 3A. FIG. 3A illustrates a schematic diagram of an architecture 300A of an auto regressive model in accordance with some embodiments of the present disclosure. As shown in FIG. 3A, unpredicted data remain masked to ensure the generation at the current position only leverages the information of previous positions. For example, the token 303 (as an example of target conformation) is generated based on the token 301 (as an example of reference conformation), token 302, masked token 304 and masked token 305, where the masked token 304 and masked token 305 are after the temporal position of the token 303. This style of auto regressive model allows full attention between all the positions to capture sequence dependencies in any order through masked token. In this way, more conformation generation tasks such as trajectory-interpolation between two conformation states may be achieved.

In some embodiments, the second update layer may be constructed based on a causal-style auto regressive model which will be introduced with reference to FIG. 3B. FIG. 3B illustrates a schematic diagram of another architecture 300B of an auto regressive model in accordance with some embodiments of the present disclosure. As shown in FIG. 3B, the temporal position of the token to be generated only attends to its previous positions, ensuring a strict auto-regressive process. For example, the token 353 (as an example of target conformation) is generated based on the masked token 350, token 351 and token 352, which are preceding the token 353.

Reference is now made back to FIG. 2, the fused feature representation (e.g., the first fused feature representation 225 and second fused feature representation 230) may contain conformation information necessary to decode three-dimensional conformation of the protein at each temporal position. Then, the fused feature representation may be used to generate the target conformation.

In some embodiments, a noisy backbone conformation 232 may be sampled from a reference distribution for the conformation sequence of the protein. A denoising operation may be performed, using a diffusion decoder 234, on the noisy backbone conformation 232 by conditioning on the first fused feature representation 225 and the second fused feature representation 230, to obtain a conformation 235 of a backbone of the protein. In some examples, the noisy backbone conformation 232 may be denoted as (Rₜ^{l}, Tₜ^{l}), where l represents the temporal position, t represents diffusion time, R represents the translation component of a conformation, and T represents the rotation component of a conformation. Then, the target conformation may be generated at least based on the conformation 235 of the backbone.

In some examples, a SE(3) diffusion model may be employed to generate the target conformation in the continuous SE(3) space. The term “SE (3)” refers to a special Euclidean group in the 3D space, describing the rigid motions composed on translations and rotations. Firstly, a noisy structure (i.e., the noisy backbone conformation 232) may be sampled from a prior distribution in SE(3). Then, the diffusion decoder 234 iteratively de-noises the noisy backbone conformation 232 through the reverse-time diffusion process. At each iteration, the intermediate noisy conformation is passed through the diffusion decoder 234 to predict a “clean” conformation (i.e., the conformation 235 of the backbone of the protein) conditioned on the first fused feature representation 225 and the second fused feature representation 230, and then SE(3) score values required for the reverse sampling steps are computed.

In some embodiments, the architecture of the diffusion decoder 234 may be a three-track model including layers of invariant point attention and transformer, to collectively update the noisy backbone conformation 232, the first fused feature representation 225 and the second fused feature representation 230.

In some embodiments, respective torsional angles for an oxygen atom in the backbone and atoms in a sidechain of the protein may be determined and a conformation of the sidechain may be determined based on the respective torsional angles. In some examples, 7 torsional angles (ϕ, φ, ω, [symbol] ₁, . . . , [symbol] ₄) may be predicted using AngleResNet for the coordinates of backbone oxygen atom and side chain atoms. Then, the target conformation may be generated based on the conformation 235 of the backbone and the conformation of the sidechain. In this way, the conformation generation model 110 may learn from different perspectives and tasks from MD trajectory data, the overall model understanding and ability may be improved, thereby benefitting multiple areas such as structural biology, drug discovery, bioengineering and the like.

In some embodiments, the sequence feature representation 205 may be determined using the first encoder 212, and the conformation feature representation 210 may be determined using the first encoder 212 and the second encoder 214. The fused feature representation may be updated at least using an auto-regressive model.

In order to train the first encoder 212, the second encoder 214 and the auto-regressive model, a loss function may be constructed firstly. A first loss component (also referred to as a target score value) may be determined based on a noisy backbone conformation from a forward diffusion process and a reference backbone conformation of the protein and a second loss component (also referred to as a model predicted score value) may be determined by using a score model based on the noisy backbone conformation and the fused feature representation (e.g., including first fused feature representation and the second fused feature representation). During the forward diffusion process, noises are gradually added to a clean backbone conformation of the protein until it becomes pure noise. A noisy backbone conformation of the protein may be sampled at a step of the forward diffusion process. Then, a loss function may be determined based on the first loss component and the second loss component. For example, the loss function may be determined based on a difference between the first loss component and the second loss component.

A challenge in applying the auto-regressive language modeling to protein dynamics lies in appropriately modeling the continuous distribution of protein conformations within the discrete language modeling framework, where expressive and efficient probabilistic models similar to multinomials are not readily available. Although previous work has attempted to model protein structure as a joint distribution of discrete structural tokens, these methods exhibit suboptimal performance in protein structure prediction accuracy. Moreover, they inherently suffer from information loss due to the discretization process. To address this limitation, diffusion probabilistic models (DPM) may be leveraged to directly model the continuous conformational space of proteins, employing denoising score-matching (DSM) as an alternative loss function to the negative log-likelihood (NLL) typically used in language models. Specifically, DSM may be used to minimize the difference between p₇₄(x^{l}|AR_{ϕ}(Encoder_{φ}(x^{<l}, [symbol] , [symbol] ))) (and

$$q ( {x}^{l} \vert {x}^{< l} , , ) in Eq . ( 4 ) as follows : \qquad ( 5 )$$

$$ℒθ ( {x}^{l} , {h}^{l} ) = 𝔼 [ ∇ log {q}_{t \vert 0} ( {x}_{t}^{l} \vert {x}_{0}^{l} ) - {s}_{θ} ( {x}_{t}^{l} , {h}^{l} , t ) ]$$

where [symbol] θ represents the loss function, xₜ^{l} represents the noisy backbone conformation from a forward diffusion process, x₀^{l} represents the reference backbone conformation, h^{l} represents the output of the autoregressive language model, that is, h^{l}=AR_{ϕ}(Encoder_{φ}(x^{<l}, [symbol] , [symbol] ) ) and h^{l} includes the fused feature representation (e.g., the first fused feature representation and the second fused feature representation). ∇logq_{t|0}(xₜ^{l}|x₀^{l}) represents the first loss component, s_{θ}represents the score model and s_{θ}(xₜ^{l}, h^{l}, t) represents the second loss component which is used to estimate the conditional score ∇logq(xₜ^{l}|h^{l}).

After determining the loss function, the first encoder 212, the second encoder 214 and the auto-regressive model may be updated based on the loss function. In this way, models are directly trained in SE (3) continuous space avoids the discretization error from structural tokenization methods.

In some embodiments, the loss function may include a denoising score-matching loss function applied to a Euclidean group with translation and rotation (e.g., SE(3)). The diffusion loss (e.g., DSM) for autoregressive models may be extended to the SE(3) space for protein conformation generation. The coordinates of heavy atoms in a protein can be parameterized using the SE(3)-torsional convention and the atomic coordinates of the backbone atoms (N−C_{α}−C) define a local coordinate for each residue determined through a Gram-Schmidt process. The position of each local coordinate with respect to the global coordinate system can be characterized by its translation and rotation. The position of N residues can be represented as x=(T, R)∈SE(3)^{N}, where T∈ [symbol] ^{N×3} SO(3) denote the translation and rotation components.

Diffusion probabilistic models (DPM) can model complex distributions in continuous space through an iterative de-noising process. DPM in the SE(3) space has been applied for protein conformation generation. Let x₀=(T₀, R₀) ∈SE(3)^{N} denotes the backbone coordinates of proteins in data. The diffusion processes defined in the translation and rotation subspace add noise to corrupt the data as follows:

$$\begin{matrix}dt = - \frac{1}{2} {β}_{t} P {T}_{t} d t + \sqrt{{β}_{t}} P d {w}_{t} , \\ {dR}_{t} = \frac{d}{d t} {σ}_{t}^{2} d {w}_{t}^{S O ( 3 )} ,\end{matrix} \qquad ( 6 )$$

In Eq. (6), t∈[0, 1] denotes the diffusion time, βₜ and αₜ represent predefined time-dependent noise schedules and P represents a projection operator removing the center of mass. wₜ and wₜ^{SO(3)} represent the standard Wiener processes in [symbol] (0, I₃)^{⊗L} and [symbol] (SO(3))^{⊗L} respectively. The transition kernel of T satisfies pₜ(Tₜ|T₀)= [symbol] (Tₜ; \sqrt{αₜ}T₀, (1−αₜ)I), where αₜ=e^{-∫}₀^{t}^{_{β}}ₛ^{ds}. The transition kernel of R satisfies pₜ(Rₜ|R₀)= [symbol] [symbol] [symbol] [symbol] ₃(Rₜ; R₀, t), where [symbol] [symbol] [symbol] [symbol] ₃ represents the isotropic Gaussian distribution on SO(3).

The associated reverse-time stochastic differential equation (SDE) is as follows:

$$\begin{matrix}d {T}_{t} = P [ - \frac{1}{2} {β}_{t} {T}_{t} - {β}_{t} ∇ log {p}_{t} ( {T}_{t} ) ] d t + \sqrt{{β}_{t}} Pd {\overline{w}}_{t} , \\ {dR}_{t} = - \frac{d}{d t} {σ}_{t}^{2} ∇ log {p}_{t} ( {R}_{t} ) dt + \frac{d}{d t} {σ}_{t}^{2} d {\overline{w}}_{t}^{SO ( 3 )}\end{matrix} \qquad ( 7 )$$

where w̅ₜ and w̅ₜ^{SO(3)} denote standard Wiener processes in the reverse time.

Given a pair of clean backbone conformation (also referred to as conformation 235 of the backbone, denoted as (R₀^{l}, T₀^{l})), noisy backbone conformation 232 (denoted as (Rₗ^{l}, Tₗ^{l}) and a latent representation h^{l}, the DPM (e.g., the diffusion decoder 234) may be trained by minimizing the SE(3) DSM loss conditioned on h^{l} as follows:

$$ℒ ( θ ) = 𝔼 [ λ ( t ) {‖ {s}_{θ} ( {T}_{t}^{l} , {h}^{l} , t ) - {∇}_{{T}_{t}} log {p}_{t \vert 0} ( {T}_{t}^{l} \vert {T}_{0}^{l} ) ‖}^{2} ] + 𝔼 [ {λ}^{r} ( t ) {‖ {s}_{θ}^{r} ( {R}_{t}^{l} , {h}^{l} , t ) - {∇}_{{R}_{t}^{l}} log {p}_{t \vert 0} ( {R}_{t}^{l} \vert {R}_{0}^{l} ) ‖}^{2} ] \qquad ( 8 )$$

During inference, a reverse sampling as Eq. (7) may be performed sample the coordinates of backbone atoms. The coordinates of the backbone oxygen atom and the side chain atoms can be determined with the additional prediction of 7 torsional angles (ϕ, φ, ω, [symbol] ₁, . . . , [symbol] ₄) describing the bond rotation.

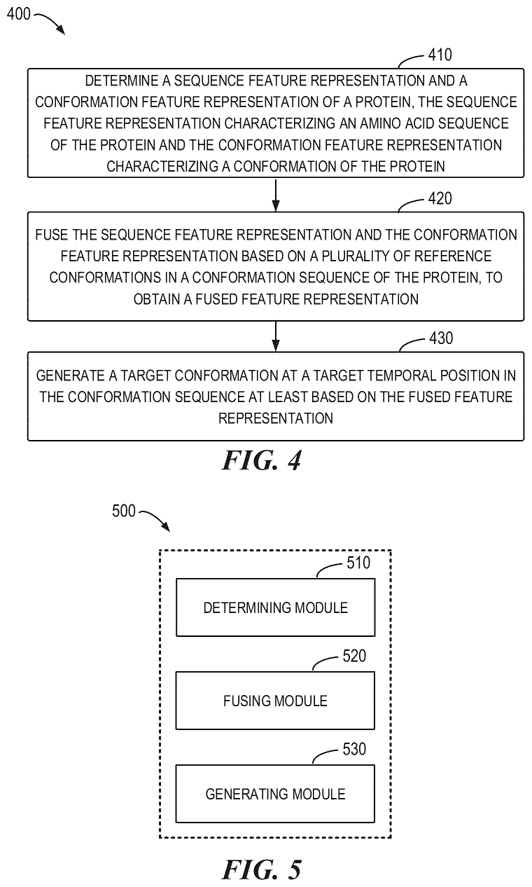

FIG. 4 illustrates a flowchart of a method 400 of conformation generation optimization in accordance with some example implementations of the present disclosure. The method 400 may be implemented at the electronic device 120 as illustrated in FIG. 1. At block 410, the electronic device 120 determines a sequence feature representation and a conformation feature representation of a protein, the sequence feature representation characterizing an amino acid sequence of the protein and the conformation feature representation characterizing a conformation of the protein. At block 420, the electronic device 120 fuses the sequence feature representation and the conformation feature representation based on a plurality of reference conformations in a conformation sequence of the protein, to obtain a fused feature representation. At block 430, the electronic device 120 generates a target conformation at a target temporal position in the conformation sequence at least based on the fused feature representation.

In some embodiments, determining the conformation feature representation comprises: generating a first conformation feature based on an amino acid protein sequence corresponding to the sequence feature representation; generating a second conformation feature from a condition for generating the target conformation, the condition comprising one of: at least one reference conformation in the plurality of reference conformations that is at a position before the target temporal position or a masked conformation; and determining the conformation feature representation by combining the first conformation feature and the second conformation feature.

In some embodiments, the sequence feature representation and the conformation feature representation are fused by a plurality of first update layers and a plurality of second update layers, and wherein fusing the sequence feature representation and the conformation feature representation comprises: updating, by the plurality of first update layers, the sequence feature representation and the conformation feature representation based on structural relations within each conformation in the plurality of reference conformations and the target conformation; and fusing, by the plurality of second update layers, the sequence feature representation and the conformation feature representation based on temporal relations across the plurality of reference conformations.

In some embodiments, the plurality of first update layers and the plurality of second update layers are arranged in an interleaving pattern.

In some embodiments, fusing, by the plurality of second update layers, the sequence feature representation and the conformation feature representation based on the temporal relations comprises: by a second update layer of the plurality of second update layers, applying an attention mechanism to the plurality of reference conformations along temporal dimension of the conformation sequence, to fuse the sequence feature representation and the conformation feature representation, wherein at least one reference conformation, of the plurality of reference conformation, that is after the target temporal position is masked.

In some embodiments, generating the target conformation comprises: sampling a noisy backbone conformation from a reference distribution for the conformation sequence of the protein; performing a denoising operation on the noisy backbone conformation by conditioning on the fused feature representation, to obtain a conformation of a backbone of the protein; and generating the target conformation at least based on the conformation of the backbone.

In some embodiments, generating the target conformation comprises: determining respective torsional angles for an oxygen atom in the backbone and atoms in a sidechain of the protein; determining a conformation of the sidechain based on the respective torsional angles; and generating the target conformation based on the conformation of the backbone and the conformation of the sidechain.

In some embodiments, the sequence feature representation is determined using a first encoder, the conformation feature representation is determined using the first encoder and a second encoder, the sequence feature representation and the conformation feature representation are updated at least using an auto-regressive model, and the method 400 is performed in training of the first encoder, the second encoder and the auto-regressive model, and the method 400 further comprises: determining a first loss component based on a noisy backbone conformation from a forward diffusion process and a reference backbone conformation of the protein; determining a second loss component by using a score model based on the noisy backbone conformation and the fused feature representation; determining a loss function based on the first loss component and the second loss component; and updating the first encoder, the second encoder and the auto-regressive model based on the loss function.

In some embodiments, the loss function comprises a denoising score-matching loss function applied to a Euclidean group with translation and rotation.

FIG. 5 shows a block diagram of an apparatus 500 for conformation generation optimization in accordance with some embodiments of the present disclosure. The apparatus 500 may be implemented, for example, or included at the electronic device 120 of FIG. 1. Various modules/components in the apparatus 500 may be implemented by hardware, software, firmware, or any combination thereof.

As illustrated, the apparatus 500 includes a determining module 510 configured to determine a sequence feature representation and a conformation feature representation of a protein, the sequence feature representation characterizing an amino acid sequence of the protein and the conformation feature representation characterizing a conformation of the protein; an fusing module 520 configured to update the sequence feature representation and the conformation feature representation based on a plurality of reference conformations in a conformation sequence of the protein, to obtain a fused feature representation; and a generating module 530 configured to generate a target conformation at a target temporal position in the conformation sequence at least based on the fused feature representation.

In some embodiments, the determining module 510 is further configured to generate a first conformation feature based on an amino acid protein sequence corresponding to the sequence feature representation, generate a second conformation feature from a condition for generating the target conformation, the condition comprising one of: at least one reference conformation in the plurality of reference conformations that is at a position before the target temporal position or a masked conformation and determine the conformation feature representation by combining the first conformation feature and the second conformation feature.

In some embodiments, the sequence feature representation and the conformation feature representation are fused by a plurality of first update layers and a plurality of second update layers. The fusing module 520 is configured to fuse, by the plurality of first update layers, the sequence feature representation and the conformation feature representation based on structural relations within each conformation in the plurality of reference conformations and the target conformation and fuse, by the plurality of second update layers, the sequence feature representation and the conformation feature representation based on temporal relations across the plurality of reference conformations.

In some embodiments, the plurality of first update layers and the plurality of second update layers are arranged in an interleaving pattern.

In some embodiments, the updating module 520 is configured to, by a second update layer of the plurality of second update layers, apply an attention mechanism to the plurality of reference conformations along temporal dimension of the conformation sequence, to fuse the sequence feature representation and the conformation feature representation, wherein at least one reference conformation, of the plurality of reference conformation, that is after the target temporal position is masked.

In some embodiments, the generating module 530 is further configured to sample a noisy backbone conformation from a reference distribution for the conformation sequence of the protein, perform a denoising operation on the noisy backbone conformation by conditioning on the fused feature representation, to obtain a conformation of a backbone of the protein and generate the target conformation at least based on the conformation of the backbone.

In some embodiments, the generating module 530 is further configured to determine respective torsional angles for an oxygen atom in the backbone and atoms in a sidechain of the protein, determine a conformation of the sidechain based on the respective torsional angles and generate the target conformation based on the conformation of the backbone and the conformation of the sidechain.

In some embodiments, the sequence feature representation is determined using a first encoder, the conformation feature representation is determined using the first encoder and a second encoder, the sequence feature representation and the conformation feature representation are updated at least using an auto-regressive model. The apparatus 500 further comprises a training module configured to determine a first loss component based on a noisy backbone conformation from a forward diffusion process and a reference backbone conformation of the protein, determine a second loss component by using a score model based on the noisy backbone conformation and fused feature representation, determine a loss function based on the first loss component and the second loss component and update the first encoder, the second encoder and the auto-regressive model based on the loss function.

In some embodiments, the loss function comprises a denoising score-matching loss function applied to a Euclidean group with translation and rotation.

FIG. 5 illustrates a block diagram of an electronic device 500 in which one or more embodiments of the present disclosure can be implemented. It would be appreciated that the electronic device 500 shown in FIG. 5 is only an example and should not constitute any restriction on the function and scope of the embodiments described herein. The electronic device 500 may be used, for example, to implement the electronic device 120 of FIG. 1. The electronic device 500 may also be used to implement the apparatus 400 of FIG. 4.

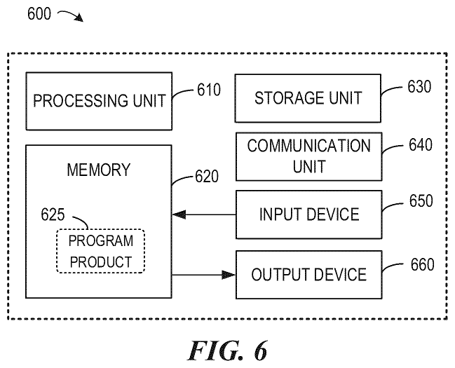

As shown in FIG. 6, the electronic device 600 is in the form of a general computing device. The components of the electronic device 600 may include, but are not limited to, one or more processing units or processors 610, a memory 620, a storage device 630, one or more communication units 640, one or more input devices 650, and one or more output devices 660. The processor 610 may be an actual or virtual processor and can execute various processes according to the programs stored in the memory 620. In a multiprocessor system, multiple processors execute computer executable instructions in parallel to improve the parallel processing capability of the electronic device 600.

The electronic device 600 typically includes a variety of computer storage medium. Such medium may be any available medium that is accessible to the electronic device 600, including but not limited to volatile and non-volatile medium, removable and non-removable medium. The memory 620 may be volatile memory (for example, a register, cache, a random access memory (RAM)), a non-volatile memory (for example, a read-only memory (ROM), an electrically erasable programmable read-only memory (EEPROM), a flash memory) or any combination thereof. The storage device 630 may be any removable or non-removable medium, and may include a machine-readable medium, such as a flash drive, a disk, or any other medium, which can be used to store information and/or data (such as training data for training) and can be accessed within the electronic device 500.

The electronic device 600 may further include additional removable/non-removable, volatile/non-volatile, transitory/non-transitory storage medium. Although not shown in FIG. 6, a disk driver for reading from or writing to a removable, non-volatile disk (such as a “floppy disk”), and an optical disk driver for reading from or writing to a removable, non-volatile optical disk can be provided. In these cases, each driver may be connected to the bus (not shown) by one or more data medium interfaces. The memory 620 may include a computer program product 625, which has one or more program modules configured to perform various methods or acts of various embodiments of the present disclosure.

The communication unit 640 communicates with a further computing device through the communication medium. In addition, functions of components in the electronic device 600 may be implemented by a single computing cluster or multiple computing machines, which can communicate through a communication connection. Therefore, the electronic device 600 may be operated in a networking environment using a logical connection with one or more other servers, a network personal computer (PC), or another network node.

The input device 650 may be one or more input devices, such as a mouse, a keyboard, a trackball, etc. The output device 660 may be one or more output devices, such as a display, a speaker, a printer, etc. The electronic device 600 may also communicate with one or more external devices (not shown) through the communication unit 640 as required. The external device, such as a storage device, a display device, etc., communicate with one or more devices that enable users to interact with the electronic device 600, or communicate with any device (for example, a network card, a modem, etc.) that makes the electronic device 600 communicate with one or more other computing devices. Such communication may be executed via an input/output (I/O) interface (not shown).

According to example implementation of the present disclosure, a computer-readable storage medium is provided, on which a computer-executable instruction or computer program is stored, where the computer-executable instructions or the computer program is executed by the processor to implement the method described above. According to example implementation of the present disclosure, a computer program product is also provided. The computer program product is physically stored on a non-transient computer-readable medium and includes computer-executable instructions, which are executed by the processor to implement the method described above.

Various aspects of the present disclosure are described herein with reference to the flow chart and/or the block diagram of the method, the device, the equipment and the computer program product implemented in accordance with the present disclosure. It would be appreciated that each block of the flowchart and/or the block diagram and the combination of each block in the flowchart and/or the block diagram may be implemented by computer-readable program instructions.

These computer-readable program instructions may be provided to the processors of general-purpose computers, special computers or other programmable data processing devices to produce a machine that generates a device to implement the functions/acts specified in one or more blocks in the flow chart and/or the block diagram when these instructions are executed through the processors of the computer or other programmable data processing devices. These computer-readable program instructions may also be stored in a computer-readable storage medium. These instructions enable a computer, a programmable data processing device and/or other devices to work in a specific way. Therefore, the computer-readable medium containing the instructions includes a product, which includes instructions to implement various aspects of the functions/acts specified in one or more blocks in the flowchart and/or the block diagram.

The computer-readable program instructions may be loaded onto a computer, other programmable data processing apparatus, or other devices, so that a series of operational steps can be performed on a computer, other programmable data processing apparatus, or other devices, to generate a computer-implemented process, such that the instructions which execute on a computer, other programmable data processing apparatus, or other devices implement the functions/acts specified in one or more blocks in the flowchart and/or the block diagram.

The flowchart and the block diagram in the drawings show the possible architecture, functions and operations of the system, the method and the computer program product implemented in accordance with the present disclosure. In this regard, each block in the flowchart or the block diagram may represent a part of a module, a program segment or instructions, which contains one or more executable instructions for implementing the specified logic function. In some alternative implementations, the functions marked in the block may also occur in a different order from those marked in the drawings. For example, two consecutive blocks may actually be executed in parallel, and sometimes can also be executed in a reverse order, depending on the function involved. It should also be noted that each block in the block diagram and/or the flowchart, and combinations of blocks in the block diagram and/or the flowchart, may be implemented by a dedicated hardware-based system that performs the specified functions or acts, or by the combination of dedicated hardware and computer instructions.

Each implementation of the present disclosure has been described above. The above description is example, not exhaustive, and is not limited to the disclosed implementations. Without departing from the scope and spirit of the described implementations, many modifications and changes are obvious to ordinary skill in the art. The selection of terms used in this article aims to best explain the principles, practical application or improvement of technology in the market of each implementation, or to enable other ordinary skill in the art to understand the various embodiments disclosed herein.

What is claimed is:

1. A method of conformation generation optimization, comprising:
determining a sequence feature representation and a conformation feature representation of a protein, the sequence feature representation characterizing an amino acid sequence of the protein and the conformation feature representation characterizing a conformation of the protein;
fusing the sequence feature representation and the conformation feature representation based on a plurality of reference conformations in a conformation sequence of the protein, to obtain a fused feature representation; and
generating a target conformation at a target temporal position in the conformation sequence at least based on the fused feature representation.\

2. The method of claim 1, wherein determining the conformation feature representation comprises:
generating a first conformation feature based on an amino acid protein sequence corresponding to the sequence feature representation;
generating a second conformation feature from a condition for generating the target conformation, the condition comprising one of: at least one reference conformation in the plurality of reference conformations that is at a position before the target temporal position or a masked conformation; and
determining the conformation feature representation by combining the first conformation feature and the second conformation feature.

3. The method of claim 1, wherein the sequence feature representation and the conformation feature representation are fused by a plurality of first update layers and a plurality of second update layers, and wherein fusing the sequence feature representation and the conformation feature representation comprises:
fusing, by the plurality of first update layers, the sequence feature representation and the conformation feature representation based on structural relations within each conformation in the plurality of reference conformations and the target conformation; and
fusing, by the plurality of second update layers, the sequence feature representation and the conformation feature representation based on temporal relations across the plurality of reference conformations.

4. The method of claim 3, wherein the plurality of first update layers and the plurality of second update layers are arranged in an interleaving pattern.

5. The method of claim 3, wherein fusing, by the plurality of second update layers, the sequence feature representation and the conformation feature representation based on the temporal relations comprises:
by a second update layer of the plurality of second update layers,
applying an attention mechanism to the plurality of reference conformations along temporal dimension of the conformation sequence, to fuse the sequence feature representation and the conformation feature representation, wherein at least one reference conformation, of the plurality of reference conformation, that is after the target temporal position is masked.

6. The method of claim 1, wherein generating the target conformation comprises:
sampling a noisy backbone conformation from a reference distribution for the conformation sequence of the protein;
performing a denoising operation on the noisy backbone conformation by conditioning on the fused feature representation, to obtain a conformation of a backbone of the protein; and
generating the target conformation at least based on the conformation of the backbone.

7. The method of claim 6, wherein generating the target conformation comprises:
determining respective torsional angles for an oxygen atom in the backbone and atoms in a sidechain of the protein;
determining a conformation of the sidechain based on the respective torsional angles; and
generating the target conformation based on the conformation of the backbone and the conformation of the sidechain.

8. The method of claim 1, wherein the sequence feature representation is determined using a first encoder, the conformation feature representation is determined using the first encoder and a second encoder, the sequence feature representation and the conformation feature representation are updated at least using an auto-regressive model, and the method is performed in training of the first encoder, the second encoder and the auto-regressive model, and the method further comprises:
determining a first loss component based on a noisy backbone conformation from a forward diffusion process and a reference backbone conformation of the protein;
determining a second loss component by using a score model based on the noisy backbone conformation and the fused feature representation;
determining a loss function based on the first loss component and the second loss component; and
updating the first encoder, the second encoder and the auto-regressive model based on the loss function.

9. The method of claim 8, wherein the loss function comprises a denoising score-matching loss function applied to a Euclidean group with translation and rotation.

10. An electronic device, comprising:
at least one processor; and
at least one memory coupled to the at least one processor and storing instructions executable by the at least one processor, the instructions, upon execution by the at least one processor, causing the electronic device to perform operations comprising:
determining a sequence feature representation and a conformation feature representation of a protein, the sequence feature representation characterizing an amino acid sequence of the protein and the conformation feature representation characterizing a conformation of the protein;
fusing the sequence feature representation and the conformation feature representation based on a plurality of reference conformations in a conformation sequence of the protein, to obtain a fused feature representation; and
generating a target conformation at a target temporal position in the conformation sequence at least based on the fused feature representation.

11. The electronic device of claim 10, wherein determining the conformation feature representation comprises:
generating a first conformation feature based on an amino acid protein sequence corresponding to the sequence feature representation;
generating a second conformation feature from a condition for generating the target conformation, the condition comprising one of: at least one reference conformation in the plurality of reference conformations that is at a position before the target temporal position or a masked conformation; and
determining the conformation feature representation by combining the first conformation feature and the second conformation feature.

12. The electronic device of claim 10, wherein the sequence feature representation and the conformation feature representation are fused by a plurality of first update layers and a plurality of second update layers, and wherein fusing the sequence feature representation and the conformation feature representation comprises:
fusing, by the plurality of first update layers, the sequence feature representation and the conformation feature representation based on structural relations within each conformation in the plurality of reference conformations and the target conformation; and
fusing, by the plurality of second update layers, the sequence feature representation and the conformation feature representation based on temporal relations across the plurality of reference conformations.

13. The electronic device of claim 12, wherein the plurality of first update layers and the plurality of second update layers are arranged in an interleaving pattern.

14. The electronic device of claim 12, wherein fusing, by the plurality of second update layers, the sequence feature representation and the conformation feature representation based on the temporal relations comprises:
by a second update layer of the plurality of second update layers,
applying an attention mechanism to the plurality of reference conformations along temporal dimension of the conformation sequence, to fuse the sequence feature representation and the conformation feature representation, wherein at least one reference conformation, of the plurality of reference conformation, that is after the target temporal position is masked.

15. The electronic device of claim 10, wherein generating the target conformation comprises:
sampling a noisy backbone conformation from a reference distribution for the conformation sequence of the protein;
performing a denoising operation on the noisy backbone conformation by conditioning on the fused feature representation, to obtain a conformation of a backbone of the protein; and
generating the target conformation at least based on the conformation of the backbone.

16. The electronic device of claim 15, wherein generating the target conformation comprises:
determining respective torsional angles for an oxygen atom in the backbone and atoms in a sidechain of the protein;
determining a conformation of the sidechain based on the respective torsional angles; and
generating the target conformation based on the conformation of the backbone and the conformation of the sidechain.

17. The electronic device of claim 16, wherein the sequence feature representation is determined at least using a first encoder, the conformation feature representation is determined at least using a second encoder, the sequence feature representation and the conformation feature representation are updated at least using an auto-regressive model, and the method is performed in training of the first encoder, the second encoder and the auto-regressive model, and the method further comprises:
determining a first loss component based on a noisy backbone conformation from a forward diffusion process and a reference backbone conformation of the protein;
determining a second loss component by using a score model based on the noisy backbone conformation and the fused conformation feature representation;
determining a loss function based on the first loss component and the second loss component; and
updating the first encoder, the second encoder and the auto-regressive model based on the loss function.

18. The electronic device of claim 17, wherein the loss function comprises a denoising score- matching loss function applied to a Euclidean group with translation and rotation.

19. A non-transitory computer readable storage medium having computer executable instructions stored thereon, the computer executable instructions, when executed by an electronic device, causing the electronic device to perform operations comprising:
determining a sequence feature representation and a conformation feature representation of a protein, the sequence feature representation characterizing an amino acid sequence of the protein and the conformation feature representation characterizing a conformation of the protein;
fusing the sequence feature representation and the conformation feature representation based on a plurality of reference conformations in a conformation sequence of the protein, to obtain a fused feature representation; and
generating a target conformation at a target temporal position in the conformation sequence at least based on the fused feature representation.

20. The non-transitory computer readable storage medium of claim 19, wherein determining the conformation feature representation comprises:
generating a first conformation feature based on an amino acid protein sequence corresponding to the sequence feature representation;
generating a second conformation feature from a condition for generating the target conformation, the condition comprising one of: at least one reference conformation in the plurality of reference conformations that is at a position before the target temporal position or a masked conformation; and
determining the conformation feature representation by combining the first conformation feature and the second conformation feature.
